(19) United States
(12) Patent Application Publication
(10) Pub. No.: US 2006/0100527 A1
(43) Pub. Date: May 11, 2006

(54) Speckle noise removal in optical coherence tomography

(76) Inventors: Giovanni Gregori, Coral Gables, FL (US); Carmen A. Puliafito, Pinecrest, FL (US); Robert W. Knighton, Miami, FL (US)

Correspondence Address: STALLMAN & POLLOCK LLP, 353 SACRAMENTO STREET, SUITE 2200, SAN FRANCISCO, CA, 94111, US

(21) Appl. No.: 11/244,244

(22) Filed: Oct. 5, 2005

Related U.S. Application Data
(60) Provisional application No. 60/622,132, filed on Oct. 26, 2004.

(51) Int. Cl. A61B 6/00

(52) U.S. Cl. 600/476

(57) ABSTRACT

A system, method and apparatus for speckle noise removal based upon structural correlation in an OCT imaging system. In accordance with the present invention, several two- or three-dimensional OCT image scans can be acquired for processing by an adaptive algorithm. Specifically, at each image point an image intensity can be computed that quantifies a measure of dispersion of the image values in a particular direction with respect to their mean. Subsequently, a direction θ₀(x, y) can be selected which minimizes the energy function at the given pixel (x, y). Finally, a value proportional to a local average of the input image around the point (x, y) can be chosen for the output image. In this way speckle noise can be minimized if not removed while, at the same time, maintaining the image substantially free of obvious artifacts.

### PRIORITY

This application claims the benefit of the filing date under 35 U.S.C.§ 119(e) of Provisional U.S. Patent Application Ser. No. 60/622,132, filed on Oct. 26, 2004, which is hereby incorporated by reference in its entirety.

### FIELD OF THE INVENTION

The present invention relates to coherent waveform based imaging, and more particularly to speckle noise removal in an optical coherence tomography (OCT) imaging system.

### BACKGROUND OF THE INVENTION

Many imaging systems utilize coherent waveforms to obtain information regarding target objects of interest. Examples include OCT, ultrasound diagnostics, and synthetic aperture radar. Randomly distributed speckle noise is an intrinsic characteristic of these types of imaging systems. Speckle noise arises from the interference that can occur between source illumination and the returning waves scattered by the microstructure of the target object. Speckle noise often takes the form of a granular pattern that degrades image quality and complicates feature analysis. Speckle noise can be a particularly substantial hurdle for automated boundary detection and segmentation techniques.

Contemporary scientific literature includes much discussion regarding the impact of speckle noise upon image quality. As such, a number of image processing approaches have been proposed whose intent is to remediate the effect of speckle noise. Optimal or near optimal strategies for specific imaging tasks under constrictive hypotheses have been proposed. Yet, in practice, there is no clearly superior and problem free approach at this time, often leaving the burden of speckle noise removal with classical noise removal algorithms such as median and gaussian filters.

Speckle noise is a serious problem in the context of OCT. OCT, as described in the publication Joseph M. Schmitt, Optical Coherence Tomography (OCT): A Review, IEEE Journal of Selected Topics in Quantum Electronics, Vol. 5, No. 4 (July/August 1999), is a relatively new imaging technique, which has demonstrated substantial imaging precision improvements over other coherent waveform imaging techniques such as ultrasound diagnostics. Today the main application of OCT is in the ophthalmic field, where it can produce sets of cross-sectional images of the human retina.

One of the techniques proposed for speckle removal is the “rotating kernel transformations” technique. First introduced in the publication, Y-K. Lee and W. Rhodes, Non-linear Image Processing by a Rotating Kernel Transformation, Optics Letters, Vol. 15, pp. 1383-1385 (December 1990), rotating kernel transformations are best described as a class of noise reducing algorithms for nonlinear image processing. Rotating kernel transformations involve the convolution of the original image with a set of operators characterized by an angular parameter, resulting in a set of processed images. At each pixel a new image value can be computed as a function of the corresponding pixel values of the convolution outputs.

Mathematically, a rotating kernel class of transformations can be described as P(x, y)=g(I*K_{θ}(x, y)), where I(x, y) is the input image, K_{θ}(x, y) is the kernel corresponding to the angle θ, g is a given function, and * reflects the convolution operator. The properties of the enhanced image P(x, y) depend upon the choices of functions g and K_{θ}. In the Lee and Rhodes publication, the proposed transformation had the form

$$g ( x , y , θ ) = {max}_{θ} ( x , y ) - {min}_{θ} ( x , y ) .$$

In this regard, the choices of these functions appear to enhance the contrast of straight-line features in the original image. When the function g is chosen to be only the maximum over all directions, the transformation is sometimes referred to as the “Sticks” algorithm.

Interestingly, in the publication R. N. Czerwinsky, D. L. Jones and W. D. O'Brien Jr., Line and Boundary Detection in Speckle Images, IEEE Transactions on Image Processing, Vol. 7, pp. 1700-1714 (December 1998), it is shown that the Sticks algorithm when applied to ultrasound speckle, leads to a near optimal detection rule for lines. Based upon the Czerwinsky publication, the publication J. Rogowska and M. E. Brezinski, Evaluation of the Adaptive Speckle Suppression Filter for Coronary Optical Coherence Tomography Imaging, IEEE Transactions on Medical Imaging, Vol. 19, pp. 1261-1266 (December 2000) proposed the use of the Sticks algorithm as a speckle reduction filter for preprocessing images obtained by OCT imaging. See also “Detection of Lines and Boundaries in Speckle Images-Application to Medical Ultrasound,” Czerwinski, et al, IEEE Trans. Medical Imaging, Vol. 18, No. 2, February 1999. All of the above articles are incorporated herein by reference.

While the Sticks algorithm performs well at detecting lines in the presence of speckle noise, it was not intended to be a speckle noise reduction filter. In this regard, the Sticks algorithm is known to be a biased estimator as the Sticks algorithm always selects the maximum direction. Furthermore the Sticks algorithm can create artifacts by enhancing spurious linear features. These artifacts can have a serious negative impact on image quality. The large amount of speckle noise present in OCT images is particularly troubling for automated image analysis. For instance the problem of segmenting and quantifying reliably the various layers present in the retina becomes extremely difficult to solve. Thus, a speckle noise reduction filter would be desirable which is not biased and which can perform well in OCT imaging.

### SUMMARY OF THE INVENTION

The present invention is a speckle noise removal method, system and apparatus configured to address the foregoing deficiencies of coherent waveform imaging. In particular, what is provided is a novel and non-obvious method, system and apparatus for speckle noise removal which accounts for the high degree of structural correlation of target features when compared to the uncorrelated nature of speckle noise in some imaging modalities. In accordance with the present invention, a speckle noise removal method can include the step of acquiring an image having a multiplicity of image points. For instance, the acquiring step can include acquiring a two-dimensional image or a three-dimensional image. Optionally, the acquiring step can include acquiring an OCT image having a multiplicity of cross-sectional images of a human retina.

Once the image has been acquired, the method can include the step of computing at each of the image points an energy that quantifies a measure of dispersion of image values in a particular direction with respect to a mean for the image values. Subsequently, the method can include selecting a direction θ₀(x, y) which minimizes energy at a given pixel (x, y). Finally, the method can include the step of determining an average of the image values in the selected chosen direction to represent a value of an output image at the point (x, y).

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a schematic illustration of an OCT imaging system configured for speckle noise removal in accordance with the present invention; and,

FIG. 2 is a flow chart illustrating a process for speckle noise removal in the OCT imaging system of FIG. 1.

### DETAILED DESCRIPTION OF THE INVENTION

The present invention is a system, method and apparatus for speckle noise removal. In accordance with the present invention, two or three-dimensional images can be acquired for processing by a novel adaptive algorithm. Specifically, at each image point an energy function can be computed that quantifies a measure of dispersion of the image values in a particular direction with respect to their mean. Subsequently, a direction θ₀(x, y) can be selected which minimizes the energy function at the given pixel (x, y). Finally, a suitable average of the image values in the chosen direction will represent the value of the output image at the point (x, y). The system, apparatus and method of the invention can be particularly effective on images like those obtained using ophthalmic OCT systems which produce sets of cross-sectional images of the human retina.

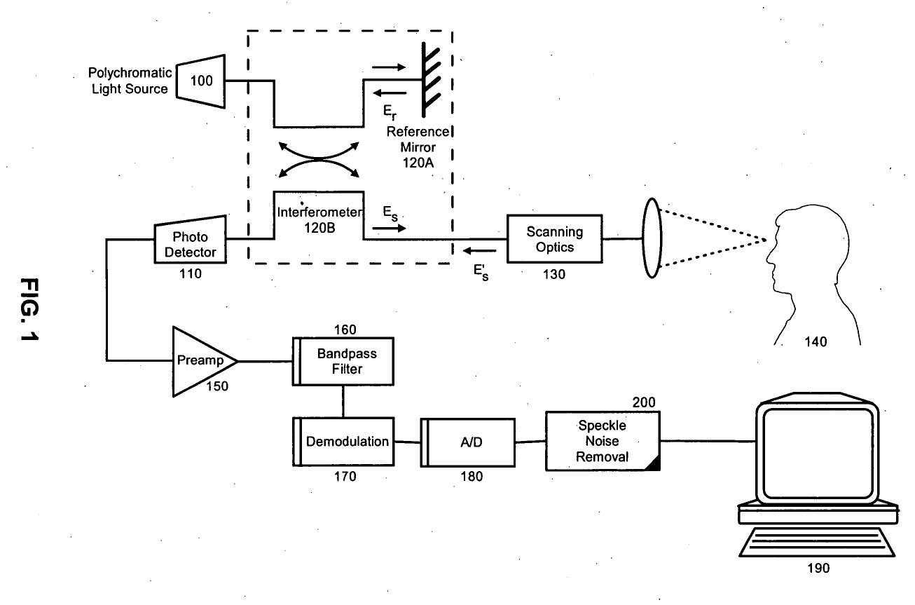

FIG. 1 depicts an OCT imaging system configured for speckle noise removal. The system can include a low coherence light source 100 such as a polychromatic light source coupled to a Michelson interferometer 120A/120B arranged for OCT imaging. The Michelson interferometer 120 can further be coupled to scanning optics 130 and a photo-detector 110 for scanning images of the tissue of a target 140, such as a retina in the case of ophthalmic OCT imaging. The signal produced by the scanning action of the scanning optics 130 can be processed first in a pre-amplifier 150, followed by a bandpass filter 160, demodulator 170 and analog to digital converter 180 as is well-known in the art of OCT. Importantly, prior to rendering an image in a computing system 190, structural correlation based speckle noise removal logic can process the digital signal to remove speckle noise in the image.

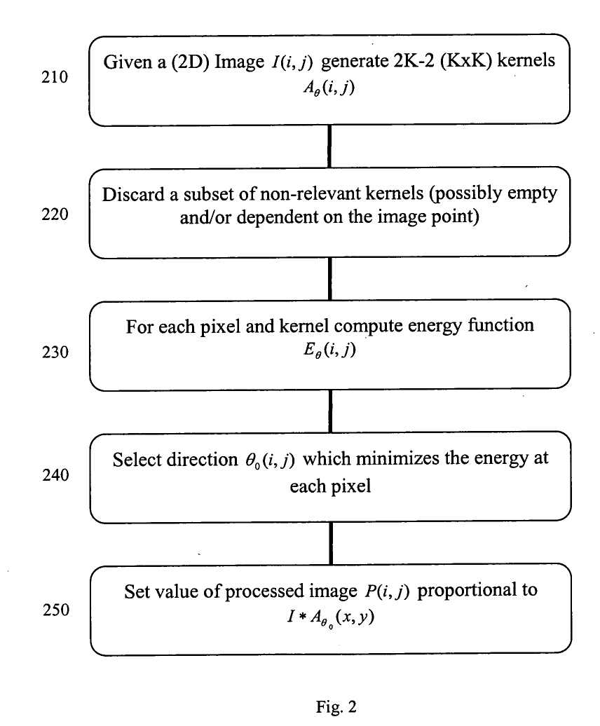

The structural correlation based speckle removal logic can be a locally adaptive filter configured for processing OCT images. FIG. 2 is a flow chart illustrating a process for speckle noise removal in the OCT imaging system of FIG. 1. For simplicity we illustrate here the method on a two dimensional image. A skilled artisan will easily extend the method to apply to three dimensional images. For a given image of N by M pixels where N is greater than or equal to M, and further given a choice of the length parameter K and the thickness parameter T, where K is greater than or equal to the value three, but less than the value of M, where K is odd, and where K is greater than T which is greater than or equal to one, a set of 2K−2 different kernels each of dimension K by K can be generated representing segments of length K and thickness T in different directions (step 210).

In a particular aspect of the invention discussed herein, the coefficients of the kernels can be set to the value one on the segment and zero otherwise (step 220). Those skilled in the art will recognize that other coefficient choices are possible and useful in specific situations. Subsequently, in block 230, an energy function is computed using the image I(i, j) and the kernels A_{θ}(i, j).

Mathematically speaking, if n_{θ} is the number of non-zero elements in A_{θ}, we can define direction-dependent image B_{θ}(i₀, j₀, i, j) and directional average of the image S_{θ}(i₀, j₀) using the following two equations
B_{θ}(i₀,j₀,i,j)=I(i,j)A_{θ}(i−i₀+(K−1)/2,j−j₀+(K−1)/2). (1)
and

$${n}_{θ} {S}_{θ} ( {i}_{0} , {j}_{0} ) = {\sum }_{i} {\sum }_{j} {B}_{θ} ( {i}_{0} , {j}_{0} , i , j ) . \qquad ( 2 )$$

As such, for each image pixel and each kernel, an energy function E_{θ}(i, j) can be defined for instance as the variance of the image values over the corresponding segment added to the weighted difference of the variances to the right and left of the pixel under consideration. As a result the energy function can be expressed mathematically as:

$${E}_{θ} ( {i}_{0} , {j}_{0} ) = \frac{{\sum }_{i} {\sum }_{j} {{B}_{θ} ( {i}_{0} , {j}_{0} , i , j )}^{2}}{{n}_{3}} - {{S}_{θ} ( {i}_{0} , {j}_{0} )}^{2} + {c}_{1} \vert \frac{{\sum }_{i} {\sum }_{j > {j}_{0}} {{B}_{θ} ( {i}_{0} , {j}_{0} , i , j )}^{2}}{{n}_{1}} - \frac{{\sum }_{i} {\sum }_{j < {j}_{0}} {{B}_{θ} ( {i}_{0} , {j}_{0} , i , j )}^{2}}{{n}_{2}} \vert + {c}_{2} ( θ ) \qquad ( 3 )$$

where c₁ is a non-negative constant, c₂ is a given function of the direction θ, and n₁, n₂, n₃ are the number of non-zero terms in the sums in the respective numerators. In block 240, for each image pixel, a direction θ₀(i, j) that minimizes the energy function E_{θ}(i, j) is determined.

Finally, in block 250 the value of the processed image P(i, j) at each pixel can be assigned to be the average in the direction that minimizes the energy. As presented herein, the structural correlation based speckle noise removal process can depend upon four parameters which can be independently chosen. These parameters include the kernels size (length K and thickness T), and the constants c₁, c₂ in the energy formulation. Other choices of energy are possible, their main feature being that they are functions of the B_{θ} measuring a weighted dispersion of the image values. The energy in a given direction θ can be a function measuring the variance of the image values along that direction. In addition the energy can contain a term weighing the difference of the partial variances computed considering only the image points to the right and the left of the center pixel.

There may be various alternatives in terms of practicing the present invention. For example, in block 220, we include the option of restricting the subset of relevant kernels, possibly in a pixel-dependent fashion. This could, for instance, make the computation substantially less intensive by selecting only a subgroup of the image points to which the invented speckle reduction algorithm can be applied and such a subgroup can correspond to the boundary region of a tissue layer that one desires to identify. Such a subset of relevant kernels can, for example, in places be empty, be only the horizontal kernel, or only the horizontal and vertical kernels, or the horizontal and near-horizontal kernels, or every kernel except the vertical one, or other subsets. In addition, the equivalent energy function E_{θ}(i, j) of Equation (3), as described in block 230 of FIG. 2, can be evaluated using the absolute value of the direction-dependent image B_{θ}(i₀,j₀,i,j), i.e. |B_{θ}(i₀,j₀, i,j)|, instead of the squared value of the direction-dependent image, i.e. B_{θ}(i₀,j₀,i,j)², and the absolute value of the directional average of the image S_{θ}(i₀,j₀),_{,} i.e. |S_{θ}(i₀,j₀)|, instead of S_{θ}(i₀,j₀)², and this will also substantially reduce the amount of computation required.

In particular, one can selectively evaluate the energy function only in complex areas of the image. In homogeneous regions, the “c₁” term in the energy function will tend to be low for the minimum-energy orientation. If the minimum “c₁” term falls below a certain threshold that is to be determined adaptively, the same kernel orientation can be used throughout the neighborhood of that point, thus saving us from having to repeatedly compute the energy function in this region. Alternatively, cross-correlation can be computed over local regions to determine homogeneity, which can determine how frequently the energy function needs to be computed. Furthermore, other metrics of homogeneity well known to those skilled in the art could also be used to determine the spatial resolution of the calculation of the energy function.

The strengths of the methodology of the present invention will be apparent to the skilled artisan. Specifically, the operation of the present invention can serve to eliminate speckle noise to a remarkable extent, without introducing obvious artifacts typically associated for instance with the use of Sticks algorithm based speckle removal logic. Moreover, the processed image maintains an altogether more pleasing aspect, in large part due to the retention of some high frequencies content. In sum, the present invention shows great potential for producing better quality OCT images, both for qualitative clinical interpretation and as a pre-processing step to automated boundary detection and other quantitative analysis. The present invention yet further can be effective in analyzing other similar forms of coherent imaging, such as medical ultrasound.

The method of the present invention can be realized in hardware, software, or a combination of hardware and software. An implementation of the method of the present invention can be realized in a centralized fashion in one computer system, or in a distributed fashion where different elements are spread across several interconnected computer systems. Any kind of computer system, or other apparatus adapted for carrying out the methods described herein, is suited to perform the functions described herein.

A typical combination of hardware and software could be a general purpose computer system with a computer program that, when being loaded and executed, controls the computer system such that it carries out the methods described herein. The present invention can also be embedded in a computer program product, which comprises all the features enabling the implementation of the methods described herein, and which, when loaded in a computer system is able to carry out these methods.

Computer program or application in the present context means any expression, in any language, code or notation, of a set of instructions intended to cause a system having an information processing capability to perform a particular function either directly or after either or both of the following a) conversion to another language, code or notation; b) reproduction in a different material form. Significantly, this invention can be embodied in other specific forms without departing from the spirit or essential attributes thereof, and accordingly, reference should be had to the following claims, rather than to the foregoing specification, as indicating the scope of the invention.

We claim:

1. A speckle noise removal method comprising the steps of:
acquiring an image comprising a plurality of image points;
computing at each of a plurality of said image points an energy function that quantifies a measure of dispersion of image values in a plurality of directions with respect to a mean for said image values;
selecting a direction θ₀(x, y) which minimizes energy function at a given pixel (x, y);
determining an average of said image values in said selected chosen direction to represent a value of an output image at the point (x, y).

2. The method of claim 1, wherein said acquiring step comprises the step of acquiring a two-dimensional image comprising points in a two-dimensional image.

3. The method of claim 1, wherein said acquiring step comprises the step of acquiring a three-dimensional image comprising points in a three-dimensional image.

4. The method of claim 1, wherein said acquiring step comprises the step of acquiring an ophthalmic coherence tomography (OCT) image comprising a plurality of cross-sectional images of a human retina.

5. The method of claim 1, further including the step of defining a set of kernels at selected image points, said kernels representing directions of interest.

6. The method of claim 5, wherein said energy function is computed at an image point for a set of kernels corresponding to all directions.

7. The method of claim 5, wherein said energy function is computed at an image point using a subset of kernels corresponding to less than all direction.

8. The method of claim 7, wherein said subset of kernels includes only horizontal and vertical kernels.

9. The method of claim 7, wherein said subset of kernels includes only horizontal and near-horizontal kernels.

10. The method of claim 7, wherein said subset of kernels includes all kernels except the vertical kernel.

11. The method of claim 7, wherein said subset of kernels at some selected image points is different than the subset of kernels at other image points.

12. The method of claim 1, wherein said energy computation is carried out only in selected areas of the image

13. The method of claim 1, further including the steps of:
evaluating the homogeneity of the image points in a region;
using the same selected direction (θ₀(x, y)) in the determining step for points in the region of homogeneity without performing the computing step for that point.

14. The method of claim 13, wherein the step of evaluating the homogeneity of the image points is performed by determining if an attribute of the energy function is below a predetermined minimum

15. The method of claim 13, wherein the step of evaluating the homogeneity of the image points is performed by cross-correlating over the selected region.
